(19) United States
(12) Patent Application Publication
(10) Pub. No.: US 2012/0016393 A1
(43) Pub. Date: Jan. 19, 2012

(54) INSTRUMENTATION AND METHOD FOR PERFORMING A MULTISTAGE GASTRIC BYPASS

(75) Inventors: Michael A. Murray, Bellevue, KY (US); Michele D'Arcangelo, Roma (IT); Alessandro Pastorelli, Roma (IT); Federico Bilotti, Aprilia, LT (IT)

(73) Assignee: ETHICON ENDO-SURGERY, INC., Cincinnati, OH (US)

(21) Appl. No.: 13/260,607

(22) PCT Filed: Apr. 10, 2009
(86) PCT No.: PCT/EP2009/054346

§ 371 Date: Sep. 27, 2011

(51) Int. Cl. A61B 17/08

(52) U.S. Cl. 606/153

(57) ABSTRACT

1. Instrumentation for carrying out a gastrointestinal bypass comprising:
means (4, 6, 12) for creating a gastric sleeve (21, 22) from the lesser curvature (13) of the stomach (1),
means for creating a gastrointestinal bypass involving said gastric sleeve (21, 22), in which said means for creating a gastrointestinal bypass include:
means for creating a gastroenteroanastomosis (26) between two closely approximated gastric and intestinal tissue portions, maintaining the continuity of the intestinal duct after the creation of the gastroenteroanastomosis,
means for creating an enteroenteroanastomosis (29) between two closely approximated tissue portions (27, 28) of the intestine, maintaining the continuity of the intestinal duct after the creation of the enteroenteroanastomosis (29),
means for sectioning the intestine (20) between the gastroenteroanastomosis (26) and the enteroenteroanastomosis (29).

In general terms, the present invention regards an instrumentation and a method for carrying out a gastrointestinal bypass. Such instrumentation and method are particularly adapted for being used laparoscopically or endolumenally or in a combined laparoscopic-endolumenal approach.

Surgical techniques are known for carrying out gastrointestinal bypasses, particularly the gastric bypass known as “Roux-en-Y-gastric bypass” (RYGB), which includes the creation of a gastric pouch and a gastroenteroanastomosis so that a large part of the stomach and intestine is bypassed by the food passing through the digestive tract. This known technique, however, has several drawbacks both in terms of high time required in execution and in terms of post-operative risks and complications.

As a matter of fact, severely obese patients with high body mass index, e.g. BMI>60 and life threatening co-morbidity would, on one hand side, gain direct benefit from bariatric surgery but, on the other hand these patients often carry an operative risk of morbidity and mortality, rendering them ineligible for bariatric surgery.

Further known but less invasive techniques for modifying the gastro-intestinal tract comprise laparoscopic sleeve gastrectomy (LSG) and Magenstrasse and Mill Procedure (MM), both involving the creation of a complete (LSG) or partial (MM) gastric sleeve, i.e. e long narrow tube obtained from the lesser curvature of the stomach. However, even though these techniques are expected to be better tolerated also by severely obese patients and obese patients with co-morbidity, they usually produce less weight loss than a Roux-en-Y-gastric bypass (RYGB) so that LASG and MM are often not satisfactory for severely obese patients.

It is further known that laparoscopic and intraluminal approaches may considerably limit the drawbacks of conventional surgical procedures. In particular, laparoscopic and intraluminal surgical procedures are usually much less invasive than traditional open surgery, thereby reducing the risks for the patient and shortening the post-operative course.

The currently available instrumentation is not yet considered sufficient to enable the creation of a gastrointestinal bypass with considerably less side effects and risks for the patient, so that the reduction of invasiveness of the available procedures and instrumentation for bariatric surgery remains a primary challenge both for surgeons and for medical device developers.

Some specific examples of critical aspects of the Roux-en-Y-gastric bypass” (RYGB) and related use of specific instrumentation are the preliminary section of the intestine, which requires considerable experience to evaluate the correct length at which to carry out the transsection of the gastric wall, the large area involved in the surgical intervention which substantially covers the entire abdomen of the patient.

The object of the present invention is therefore to propose an instrumentation and a method for creating gastrointestinal bypasses, thereby obviating the drawbacks mentioned with reference to the prior art.

Within the above general object, a specific object of the present invention is to provide an instrumentation and a method for carrying out bariatric surgery tolerable by severely obese patients or by obese patients with life-threatening co-morbidity (which would not be eligible for RYGB) and suitable to obtain a considerable and long-lasting weight loss as necessary for this category of patients.

These and other objects are achieved by an instrumentation for carrying out a gastrointestinal bypass in accordance with annexed claim 1 and by a method for carrying out a gastrointestinal bypass in accordance with claim 6.

In accordance with an aspect of the present invention, an instrumentation for carrying out a gastrointestinal bypass comprises:
means for creating a gastric sleeve from the lesser curvature of the stomach,
means for creating a gastrointestinal bypass involving said gastric sleeve, in which said means for creating a gastrointestinal bypass include:
   means for closely approximating a first tissue portion of the intestine with a second tissue portion of the gastric sleeve, creating a first loop of the intestine between the gastric sleeve and the first tissue portion,
   means for creating a gastroenteroanastomosis between the closely approximated first and second tissue portions, maintaining the continuity of the intestinal duct after the creation of the gastroenteroanastomosis,
   means for closely approximating an additional first tissue portion of the intestine arranged distally (or in other words: downstream with respect to the natural flow along the intestinal duct) from the gastroenteroanastomosis and an additional second tissue portion of the intestine arranged proximally (or in other words: upstream with respect to the natural flow along the intestinal duct) from the gastroenteroanastomosis, creating a second loop of the intestine arranged distally from the gastroenteroanastomosis, wherein these means are configured to position the additional second tissue portion of the intestine proximally from and close to the gastroenteroanastomosis,
   means for creating an enteroenteroanastomosis between the two closely approximated additional first and additional second tissue portions of the intestine, maintaining the continuity of the intestinal duct after the realization of the enteroenteroanastomosis,
   means for transecting the intestine between the gastroenteroanastomosis and the enteroenteroanastomosis.

In accordance with a further aspect of the present invention, a method for carrying out a gastrointestinal bypass comprises the steps of:
creating a gastric sleeve from the lesser curvature of the stomach,
closely approximating a first tissue portion of the intestine to a second tissue portion of the gastric sleeve, thereby creating a first loop of the intestine between the gastric sleeve and the first tissue portion,
creating a gastroenteroanastomosis between the closely approximated first and second tissue portions,
maintaining the continuity of the intestinal duct after the creation of the gastroenteroanastomosis,
closely approximating an additional first tissue portion of the intestine arranged distally from the gastroenteroanastomosis (or in other words: downstream with respect to the natural flow along the gastrointestinal tract) with an additional second tissue portion of the intestine arranged proximally (or in other words: upstream with respect to the natural flow along the gastrointestinal tract) from the gastroenteroanastomosis, thereby creating a second loop of the intestine which is distal with respect to the gastroenteroanastomosis, wherein said additional second tissue portion of the intestine is positioned close to the gastroenteroanastomosis,
creating an enteroenteroanastomosis between the closely approximated additional first and additional second tissue portions of the intestine,
maintaining the continuity of the intestinal duct after the creation of the enteroenteroanastomosis,
transecting the intestine between the gastroenteroanastomosis and the enteroenteroanastomosis.

Further characteristics and advantages of the instrumentation and method for carrying out a multistage gastrointestinal bypass according to the invention will be apparent from the following description of non limiting exemplary embodiments, with reference to the attached figures, in which:

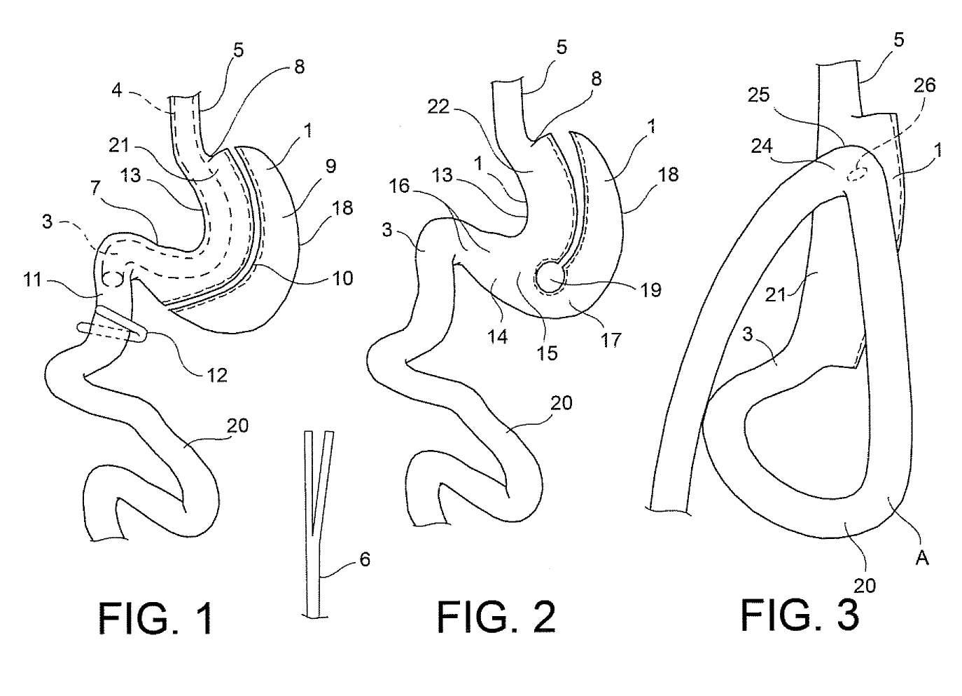

FIG. 1 illustrates a laparoscopic sleeve gastrectomy (LSG) for the creation of a gastric sleeve from the lesser curvature of the stomach as a first phase of a multistage method for carrying out a gastrointestinal bypass in accordance with one embodiment of the present invention.

FIG. 2 illustrates a laparoscopic Magenstrasse and Mill procedure (MM) for the creation of a gastric sleeve of the “Magenstrasse” type (“street of the stomach”), which is also a long narrow tube fashioned from the lesser curvature of the stomach, as a alternative first phase of the multistage method for carrying out a gastrointestinal bypass in accordance with an embodiment of the invention.

FIG. 3 illustrates a first step (“first loop creation”) of a creation of a gastrointestinal bypass as a subsequent phase or additional phase of the multistage method in accordance with an embodiment of the invention, wherein FIG. 3 illustration is exemplarily based on the first phase embodiment of FIG. 1 (LSG), but could analogously applied to the first phase embodiment of FIG. 2 (MM).

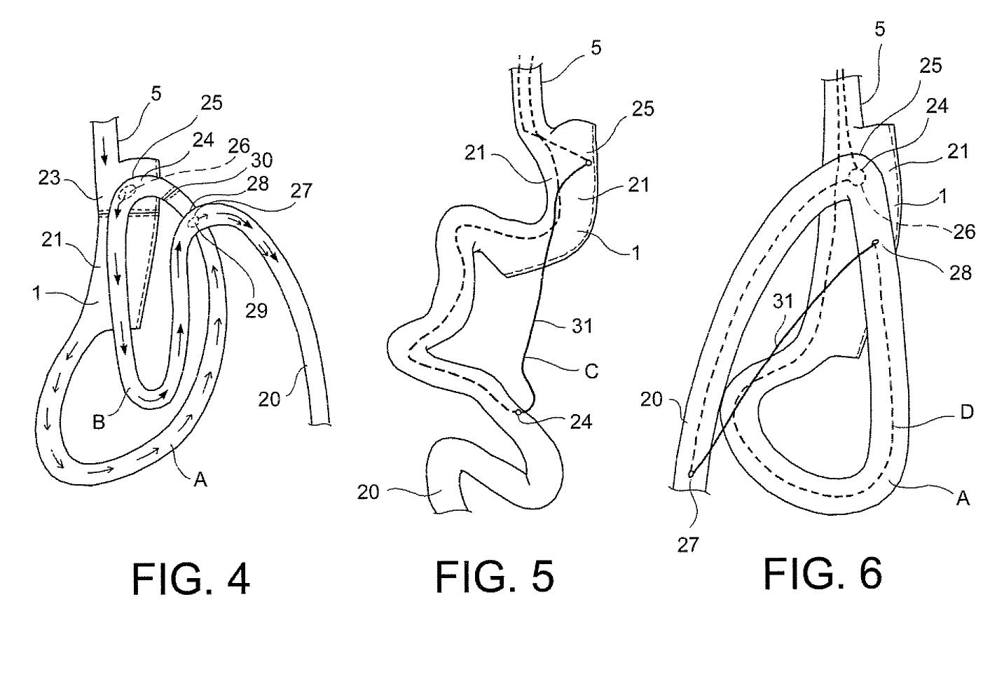

FIG. 4 illustrates a second step (“second loop creation”) of the additional phase of the multistage method in accordance with an embodiment of the invention, wherein FIG. 4 illustration is exemplarily based on the first phase embodiment of FIG. 1 (LSG), but could analogously applied to the first phase embodiment of FIG. 2 (MM).

FIGS. 5-8 illustrate several steps of the additional phase of the multistage method in accordance with an embodiment of the invention, said figures being exemplarily based on the first phase embodiment of FIG. 1 (LSG), but could analogously applied to the first phase embodiment of FIG. 2 (MM).

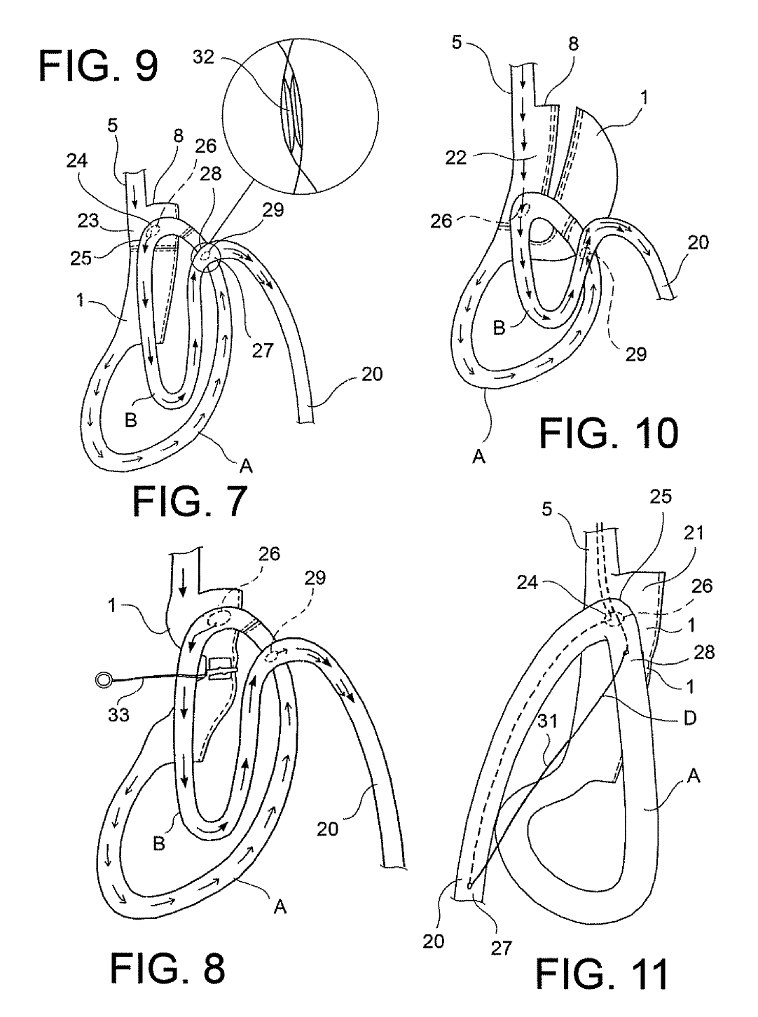

FIG. 9 illustrates an enlarged detail of FIG. 7 according to one possible embodiment and from a different viewpoint.

FIG. 10 illustrates the additional phase configuration of FIG. 7 based on the first phase embodiment of FIG. 2 (MM).

FIG. 11 illustrates a possible embodiment variation of FIG. 4.

With reference to the figures in which like reference numerals denote like anatomical structures and device features, the present invention concerns a multistage method for carrying out a gastrointestinal bypass, comprising:

A) a first phase of creating a gastric sleeve aimed to obtain a preliminary morphological and, hence, functional modification of the digestive tract by means of comparatively low-invasive surgery which is tolerated also by severely obese patients or by obese patients with co-morbidity, which would be otherwise ineligible for bariatric surgery and particularly for LRYGB,

B) a waiting phase, in which the patient experiences a preliminary loss of weight due to the effect of the gastric sleeve, thereby improving the general conditions of the patient so that he can face an additional bariatric surgery with a significantly lower risk of morbidity or mortality,

C) an additional phase of carrying out a gastrointestinal bypass in a significantly low invasive way, aimed to a more significant and long-lasting loss of weight of the patient.

For the sake of clarity, the following embodiment description is subdivided in a first part dealing with the instrumentation and method concerning the first phase of creating a gastric sleeve and a second part dealing with the instrumentation and method concerning the additional phase of carrying out a gastrointestinal bypass.

### A) Instrumentation and Method Concerning the Phase of Creating a Gastric Sleeve

With reference to FIG. 1, a gastric sleeve 1 is created by laparoscopic sleeve gastrectomy. The patient is given anesthesia and an endopath gasless technique can be used to enter the abdomen by a trocar, preferably on the supraumbelical lateral border of the right rectus, further trocars can be placed in subxyphoid, right costal margin, left costal margin, lateral border of the left rectus positions and an additional trocar for visualization means, such as a camera, can be inserted in the abdominal cavity.

Alternatively, a single-site abdominal access method may be used in which a single access device with multiple ports is inserted into the abdomen.

The greater curvature 18 of the stomach 1 which is destined to be excised, must be isolated from blood supply. This can be obtained by dividing the vascular supply of the greater curvature 18 of the stomach 1 with a harmonic scalpel, electrocautery dividing device or a stapling dividing device starting from the left crus of the diaphragm and proceeding distally to the pylorus 3.

Before commencing the gastrectomy, a gastric tube 4, preferably a naso-gastric tube is endolumenally inserted through the nose and the esophagus 5 into the stomach 1 and with its tip down into the duodenum to define the shape and dimension of the gastric sleeve 21.

Alternatively, an endoscope or bougie may be used orally.

Subsequently, a laparoscopic linear stapler 6 can be used to staple and contemporaneously cut the stomach 1 close to the naso-gastric tube 4, preferably starting at about 2 cm from the pylorus 3 up to the incisura angularis 7 and further up to the angle of HIS 8. During stapling, different types of linear laparoscopic staplers 6 with different staple line lengths can be used, as required by the space and access conditions.

As a result, the gastric sleeve 21 or, in other words: gastric tube, is obtained and the remaining part 9 of the stomach 1 is excised. The staple line 10 can be completed by placing additional sutures.

After the completion of the gastrectomy, a leak test can be performed using e.g. methyline blue dye. To this end, the duodenum 11 can be clamped with a laparoscopically applied clamp 12 and the gastric sleeve 21 irrigated with the dye. The excised part 9 of the stomach 1 can be removed through one of the openings created for the trocars, which might be additionally enlarged for this purpose.

In accordance with an alternative embodiment, illustrated in FIG. 2, a gastric sleeve 22 or tube can be created by means of a so called Magenstrasse (“street of the stomach”) and Mill Operation (MM). Such a gastric sleeve 22 is configured as a long narrow tube fashioned from the lesser curvature 13 of the stomach which conveys food from the esophagus 5 to the antral mill 14, with the advantage that normal antral grinding of solid food and antro-pyloro-duodenal regulation of gastric emptying and secretion are preserved.

In accordance with this embodiment, a midline epigastric incision and mechanical upward retraction of the rib cage or, alternatively, a plurality of laparoscopic access ports or a single-site access device could be used to provide access to the upper stomach and esophagus. A gastric tube 4, preferably a naso-gastric tube is endoluminally inserted through the nose and the esophagus 5 into the stomach 1 and with its tip down into the duodenum to define the shape and dimension of the gastric sleeve 22. Alternatively, an endoscope or bougie may be used orally. A laparoscopic or open surgery circular stapler can be used to create a circular defect 19 in the gastric antrum 15, preferably close beyond the incisura angularis and about 5 to 6 cm from the pylorus 3.

Alternatively, a linear cutter designed to only staple and cut at the distal portion of the jaw assembly may be used to create a ‘button-hole’ defect in the gastric antrum.

In preparation of the division of the stomach 1 and during division thereof, the gastric tube 4 should be held firmly against the lesser curvature 13 so that the gastric sleeve 22 (in the present embodiment a gastric sleeve of the “Magenstrasse”-type) does not become substantially larger than the diameter of the gastric tube 4. To this end, a muscle relaxant drug can be used to render the stomach 1 flaccid so that it can be easier torn around the gastric tube 4. Moreover, in order to preserve the antral mill activity, at least one major terminal branch of the Latarjet nerve 16 should be preserved both on the anterior and posterior walls of the antrum 15 and a wide passage 17 must be left between the circular defect 19 and the greater curvature 18 to enable unobstructed flow of fluid from the excluded body of the stomach 1 into the antrum 15. It is therefore preferable to use a small circular stapler with a staple line diameter of about 1 cm-1.5 cm for the creation of the circular defect 19.

After removal of the circular “doughnut” of gastric wall, a laparoscopic or open-surgery linear stapler can be used to create the “Magenstrasse”-type gastric sleeve 22 by dividing the stomach 1 completely from the circular defect 19 in the antrum 15 to the angle of HIS 8 or at least proximate thereto. The free stapled edges of stomach 1 can be over-sutured with running absorbable sutures to reinforce the staple lines and ensure hemostasis.

After the completion of this only partial gastrectomy, a leak test can be performed using e.g. methyline blue dye. To this end, the duodenum 11 can be clamped with a preferably laparoscopically applied clamp 12 and the gastric sleeve 22 irrigated with the dye.

### B) Waiting Phase Between the Creation of the Gastric Sleeve and the Creation of the Gastro-Intestinal Bypass

Even though, in accordance with a possible embodiment, the creation of the gastro-intestinal bypass might be carried out substantially immediately after the creation of the gastric sleeve 21, 22, in accordance with a preferred embodiment, a waiting phase of at least 2 months, preferably of 2 months to 3 years, yet more preferably of 1 year to 2 years is provided, in which the patient experiences a preliminary loss of weight due to the effect of the gastric sleeve 21, 22, thereby improving the general conditions of the patient so that he can face an additional bariatric surgery with a significantly lower risk of morbidity or mortality,

### C) Instrumentation and Method for Carrying out a Gastrointestinal Bypass on the Gastric Sleeve

In accordance with an embodiment, the additional phase of carrying out a gastrointestinal bypass on the gastric sleeve 21, 22 comprises the steps as illustrated in FIGS. 3 and 4. Particularly, such method comprises steps of closely approximating and joining tissues to form anastomoses adapted to maintain or restore the integrity and continuity of the intestinal duct after each anastomosis formation (either gastroenteroanastomosis or enteroenteroanastomosis). Moreover, the gastroenteroanastomosis and the enteroenteroanastomosis are created at close distances, thereby reconciling the contrasting needs to limit the operating zone, for example to only one upper zone of the abdomen, while keeping a wide operating and visual area.

In accordance with a possible embodiment, a first step of the additional phase of creating a gastro-intestinal bypass contemplates the creation of a gastric pouch 23 to which the first portion of the intestine will be connected. Subsequently, an ansa of the intestine is selected to be united to the gastric pouch. The choice is operated by means of measuring the available length of intestine and possibly verifying that tensions or distortions are prevented.

A first tissue portion 24 of the intestine 20, corresponding with the chosen ansa, is therefore closely approximated to a second tissue portion 25 of the gastric sleeve 21, 22 of stomach 1 at the gastric pouch 23. A first loop A of the intestine 20 is thus created which extends between the gastric sleeve 21, 22 of stomach 1 and the first tissue portion 24 of the intestine 20. The two tissue portions 24, 25 are slightly incised, forming an enterotomy and a gastrotomy to allow the insertion of respective jaws of a linear stapler 6. One jaw of the linear stapler is inserted in the gastrotomy made at the second tissue portion 25 of the gastric sleeve 21, 22 of stomach 1. The other jaw of the linear stapler is inserted in the enterotomy made at the first tissue portion 24 of the intestine 20. One flap of the two incisions is then sutured (stapled) by the linear stapler 6 by means of a sequence of points, joining the two tissue portions 24, 25 and partially defining the gastroenteroanastomosis 26.

In accordance with a preferred embodiment, the gastrotomy and enterotomy are done before closely approximating the two tissue portions 24, 25. In this case, the linear stapler 6 is used as an instrument for bringing the two tissue portions 24, 25 close together, for example inserting one of the two jaws of the linear stapler into the enterotomy and using the linear stapler 6 for bringing the first tissue portion 24 close to the second tissue portion 25. The other jaw is then inserted into the gastrotomy so to join the two tissue portions 24, 25.

To complete the gastroenteroanastomosis 26 between the first and second tissue portions 24, 25, the flaps of the gastrotomy and enterotomy are reclosed, for example by means of suture. This allows restoring the continuity of the intestinal duct after the formation of the gastroenteroanastomosis 26. Indeed, after having completed the gastroenteroanastomosis 26, the intestinal tract 20 maintains its integrity and continuity, since the incisions carried out were restapled or sutured to form the gastroenteroanastomosis 26.

In accordance with a preferred embodiment, the step of carrying out the gastroenteroanastomosis 26 can be performed substantially at the end of the gastro-intestinal bypass procedure, before transecting the intestine 20 and before carrying out a seal test, as will be described below.

Subsequently, an additional ansa of the intestine 20 is chosen, distal with respect to the gastroenteroanastomosis 26 with reference to the natural flow along the intestinal duct, i.e. to the flow before carrying out the gastrointestinal bypass. In other words, by distal it is intended an ansa downstream of the gastroenteroanastomosis 26 with reference to the natural flow along the intestinal duct. This definition of proximal or distal will also be used below with reference to the natural flow inside the intestinal duct.

A corresponding additional first tissue portion 27 of the additional intestine ansa, distal with respect to the gastroenteroanastomosis 26 with reference to the natural flow along the intestinal duct, is then closely approximated to an additional second tissue portion 28 of the intestine, proximal from the portion 27 with respect to the natural flow along the intestinal duct, thereby realising a second loop B of the intestine 20 which is distal with respect to the gastroenteroanastomosis 26. In other words, as defined above, the additional first tissue portion 27 is arranged downstream of the gastroenteroanastomosis 26 with respect to the natural flow along the intestinal duct, whereas the additional second tissue portion 28 is arranged upstream of the gastroenteroanastomosis 26 with respect to the natural flow along the intestinal duct. The additional second tissue portion 28 of the intestine 20, proximally arranged with respect to the gastroenteroanastomosis 26, is brought close to the gastroenteroanastomosis 26. At each of the two additional tissue portions 27, 28, an incision (enterotomy) is made, adapted to receive a respective jaw of the linear stapler 6. A first jaw of the linear stapler 6 is inserted in the enterotomy made at the additional first tissue portion 27 of the intestine 20. A second jaw of the linear stapler 6 is inserted in the enterotomy made at the additional second tissue portion 28 of the intestine 20. A sequence of staples is then applied, which partially unites the flaps of the enterotomies and partially defines the enteroenteroanastomosis 29.

In accordance with a preferred embodiment described above, the enterotomies are realised before closely approximating the additional first and second tissue portions 27, 28 to one another. In this case, the linear stapler 6 is used as an instrument for approximating the additional first and second tissue portions 27, 28, for example through inserting one of the two jaws of the linear stapler 6 into the enterotomy made at the additional first distal tissue portion 27, and using the stapler for bringing the additional first distal tissue portion 27 close to the additional second proximal tissue portion 28. The other jaw is then inserted in the enterotomy of the additional second proximal tissue portion 28 in order to join the two tissue portions.

The enteroenteroanastomosis 29 between the two additional tissue portions 27, 28 is subsequently completed by reclosing the flaps of the enterotomies to restore the continuity of the intestinal duct after the creation of the enteroenteroanastomosis 29. The remaining flaps of the enterotomies are joined for example by means of suture.

In accordance with a preferred embodiment, the step of completion of the enteroenteroanastomosis 29 is carried out near the end of the procedure, at the same time as the completion of the gastroenteroanastomosis 26 before sectioning the intestine 20 and before carrying out the seal test, as will be described below.

After having completed the enteroenteroanastomosis 29, the intestinal tract maintains its integrity and continuity, since the incisions carried out were restapled to form the enteroenteroanastomosis 29.

As illustrated in FIG. 4, the gastroenteroanastomosis 26 and the enteroenteroanastomosis 29 are very close to each other, and permit reducing the operation space to the upper part of the abdomen.

After having made the second loop B, it is now possible to carry out, preferably contemporaneously, a seal test of the two anastomoses 26, 29, for example by means of methylene blue dye. The step of transecting the intestine 20 between the gastroenteroanastomosis 26 and the enteroenteroanastomosis 29 can be performed at the end of the gastro-intestinal bypass procedure, after the correct functioning and leak tightness of the two anastomoses 26, 29 was verified. In FIG. 4, the transecting of intestine 20 is indicated with a section line 30.

Advantageously, the aforesaid gastro-intestinal bypass phase of the multistage method is performed laparoscopically, comprising an initial step of inserting trocars, preferably four trocars arranged respectively in the following zones: epigastrium, left flank, and two trocars at the mesogastrium zone.

In accordance with an aspect of the present invention, the instrumentation for carrying out the gastro-intestinal bypass phase of the method comprises: means for bringing a first tissue portion 24 of the intestine 20 close to a second tissue portion 25 of the gastric sleeve 21, 22 of stomach 1, creating a first loop A of the intestine 20 between the gastric sleeve 21, 22 of stomach 1 and the first tissue portion 24 of the intestine, means for creating a gastroenteroanastomosis 26 between the two portions 24, 25 of closely approximated tissue, maintaining or restoring the continuity of the intestinal duct after the creation of the gastroenteroanastomosis 26, means for closely approximating an additional first tissue portion 27 of the intestine 20 which is distally arranged or downstream with respect to the gastroenteroanastomosis 26 with reference to the natural flow along the intestinal duct, and an additional second tissue portion 28 of the intestine 20 arranged proximally, or upstream, with respect to the natural flow along the intestinal duct, thereby creating second loop B of the intestine 20 which is distal with respect to the gastroenteroanastomosis 26, wherein said additional second tissue portion 28 of the intestine 20 arranged proximally with respect to the gastroenteroanastomosis 26 is closely approximated to the gastroenteroanastomosis 26, means for creating an enteroenteroanastomosis 29 between the two closely approximated tissue portions 27, 28 of the intestine 20, maintaining or restoring the continuity of the intestinal duct after the creation of the enteroenteroanastomosis 29, as well as means for sectioning the intestine 20 between the gastroenteroanastomosis 26 and the enteroenteroanastomosis 29.

Preferably, the aforesaid instrumentation moreover comprises means for carrying out a seal test both of the gastroenteroanastomosis 26 and the enteroenteroanastomosis 29, before the intestine 20 is sectioned between the gastroenteroanastomosis 26 and the enteroenteroanastomosis 29. Still more preferably, the means for carrying out the seal test are adapted to simultaneously test both the gastroenteroanastomosis 26 and the enteroenteroanastomosis 29. Such means may comprise, for example, means for inserting and visualising methylene blue dye through the intestinal duct.

In accordance with a possible embodiment, the means for creating the gastroenteroanastomosis 26 comprise a linear stapler 6 adapted to partially join the flaps of a gastrotomy and enterotomy respectively made at the first portion 24 of the intestine 20 and the second portion 25 of the gastric sleeve 21, 22 of stomach 1. The means for creating the gastroenteroanastomosis 26 comprise moreover means for completing the gastroenteroanastomosis 26 by reclosing the flaps still open after the use of the linear stapler 6, said means being adapted to restore the continuity of the intestinal duct after the creation of the gastroenteroanastomosis 26. Preferably, the linear stapler 6 also carries out the function of bringing close together the two tissue portions 24, 25 to be joined, inserting a jaw in the first tissue portion 24 and using the stapler as a means for transporting the first tissue portion 24 in correspondence with the second tissue portion 25.

In accordance with a possible embodiment, the means for creating the enteroenteroanastomosis 29 comprise a linear stapler 6 adapted to partially join the flaps of enterotomies respectively made at the additional first and second portions 27, 28 of the intestine 20. Moreover, the means for creating the enteroenteroanastomosis 29 comprise means for completing the enteroenteroanastomosis 29 by reclosing the flaps still open after the use of the linear stapler, said means being adapted to restore the continuity of the intestinal duct after the creation of the enteroenteroanastomosis 29. Preferably, the linear stapler also carries out the function of means for bringing close together the two additional tissue portions 27, 28 to be joined, inserting a jaw in the additional first tissue portion 27 and using the stapler as a means for transporting the additional first tissue portion 27 in correspondence with the additional second tissue portion 28.

The instrumentation according to the present invention advantageously comprises means for preliminarily creating a gastric pouch 23, wherein the second tissue portion 25 of the gastric sleeve 21, 22 of stomach is arranged at the gastric pouch 23.

Preferably, the means used are adapted to create the gastrointestinal bypass laparoscopically.

The method and the instrumentation described above may be applied both to the step of carrying out the gastroenteroanastomosis 26 and the step of carrying out the enteroenteroanastomosis 29, or to one of these steps.

The described multistage method and instrumentation allow severely obese patients and obese patients with life threatening co-morbidity to become eligible for gastric bypass surgery and assure at the same time a significant and long lasting weight loss, as well as an improvement of the general physical and clinical conditions of those patients. Moreover, the present invention makes it possible to reduce the risks of mortality with gastrointestinal bypasses and considerably limit the operation time of the gastric bypass surgery phase. Thanks to the conservation of the continuity of the intestine until the completion of the two anastomoses 26, 29, it is possible to verify the tightness of both simultaneously. Moreover, due to the close arrangement of the two anastomoses 26, 29, the operation area is limited to the upper zone of the abdomen. Additionally, the advantageous prevision of forming two intestine loops A, B without previously interrupting the continuity of the intestine 20 allows choosing the correct length, so to avoid tensions and torsion in the intestinal duct. Moreover, the creation of the gastroenteroanastomosis 26 without preliminary sectioning of the ansae permits reducing the risk of incorrectly joining the segments or inducing undesired torsions. Moreover, the combination of the present method results particularly innovative, by which the two intestine loops A, B are made foreseeing the use of linear staplers 6 for creating the anastomoses 26, 29, preferably close to each other. As a matter of fact, beyond the above mentioned advantages, the use of the linear stapler 6 permits limiting the size and extension of the bleeding, the losses and risk of stenosis. The use of the linear stapler for an application as previously described overcomes a deep-rooted disadvantage which previously prevented its application. In particular, the advantageous prevision of using a linear stapler for carrying out of both anastomoses in a method as described permits maintaining good blood perfusion of the affected tissues and permits having available an instrument of limited size adapted to operate in a restricted area of the abdomen.

In accordance with a different embodiment, the gastric bypass phase steps of closely approximating tissues and/or creating the gastroenteroanastomosis and/or enteroenteroanastomosis are performed intraluminally by using an anastomotic device as for example illustrated in FIGS. 5-8. Such device is preferably made to slide along a guide means, preliminarily inserted through the portions to be brought together and/or joined and making up part of the instrumentation according to the present invention. Preferably, the guide means comprises at least one guide wire 31 arranged as an open loop which crosses through the portions to be joined and which can be associated with an anastomotic device.

The partial realisation of a pouch 23 in the gastric sleeve 21, 22 of stomach may be previously foreseen, to which the first intestine portion 24 will be connected. Subsequently, a first guide wire open loop C is created through the open portion of the gastric pouch 23 and through the first portion 24 of the intestine 20 and the second portion 25 of the gastric sleeve 21, 22 of stomach 1 to be joined. In accordance with one possible embodiment, the gastric pouch 23 is created at the end of the procedure, after the creation of the gastroenteroanastomosis 26 and the enteroenteroanastomosis 29. In such case, the first guide wire open loop C is created through the gastric sleeve 21, 22 of stomach 1 and the intestine 20, crossing the tissue portions 24, 25 to be united as for example illustrated in FIG. 5. An anastomotic device is inserted and locked on the guide means, and transported, by means of the guide means itself, until it abuts against the first tissue portion 24 of intestine 20 to be joined and brings it close to the second tissue portion 25 of the gastric sleeve 21, 22 to be joined. This first sequence of steps concludes with the creation of a gastroenteroanastomosis 26. The anastomotic device can be embodied by means of an anvil adapted to be locked on the guide wire 31 and adapted to cooperate with a circular stapler to carry out the gastroenteroanastomosis 26. Alternatively, the anastomotic device can be a device adapted to position an anastomotic ring, preferably elastic, to keep the two tissue portions 24, 25 joined (as for example illustrated in FIG. 9 with reference to the enteroenteroanastomosis 29) or other anastomotic devices adapted for such purpose. Subsequently, a second guide wire open loop D is created through the two portions of the intestine to be joined (additional first portion 27, distal with respect to the gastroenteroanastomosis 26, and additional second portion 28, proximal with respect to the gastroenteroanastomosis 26). In case the gastric pouch 23 is partially created at the beginning of the bypass phase of the multistage procedure, the second guide wire open loop D also crosses the open portion of the gastric pouch 23. FIG. 6 illustrates the second guide wire open loop D without preliminary, partial formation of the gastric pouch 23. The second guide wire open loop D crosses the gastroenteroanastomosis 26, the additional first portion 27, distal with respect to the gastroenteroanastomosis 26 with reference to the natural flow of the intestine duct, the additional second portion 28, proximal with respect to the gastroenteroanastomosis 26, and has the two ends preferably at the same orifice, e.g. mouth or nose. FIG. 11 illustrates a possible variation wherein the guide wire open loop D is created by crossing the gastroenteroanastomosis 26, the additional first portion 27, distal with respect to the gastroenteroanastomosis 26 with reference to the natural flow of the intestine duct, the additional second portion 28, proximal with respect to the gastroenteroanastomosis 26, and has the two ends in correspondence preferably with the same orifice, e.g. mouth or nose.

Also in this case, an anastomotic device is inserted and locked on the guide means, e.g. guide wire 31, and transported, by means of the guide means itself, until it abuts against the additional first portion 27 to be joined and brings it close to the additional second portion 28 to be joined. This second sequence of steps concludes with the creation of an enteroenteroanastomosis 29 and the completion of the gastric pouch 23. Similar to the formation of the gastroenteroanastomosis 26, the anastomotic device can be embodied as an anvil adapted to be locked on the guide wire 31 and adapted to cooperate with a circular stapler to carry out the enteroenteroanastomosis 29. Alternatively, the anastomotic device can comprise a device adapted to position an anastomotic ring 32, preferably elastic, to keep the two tissue portions joined, or other anastomotic devices adapted for such purpose. After completion of the gastroenteroanastomosis 26 and the enteroenteroanastomosis 29, it is now possible to simultaneously test both anastomoses 26, 29 as previously described. Preferably, the step of the seal test is carried out after the creation (or completion) of the gastric pouch 23. Finally, the intestine 20 is sectioned and stapled between the gastroenteroanastomosis 26 and the enteroenteroanastomosis 29 along transecting line 30. FIG. 7 illustrates this latter situation wherein also the creation of the gastric pouch 23 is highlighted at the end of the procedure. FIG. 9 illustrates a detail of FIG. 7 wherein a possible formation is highlighted of the enteroenteroanastomosis 29 by means of a preferably elastic ring device deployed by means of an anastomotic positioning device. The same solution may possibly be adopted for the gastroenteroanastomosis 26. FIG. 8 illustrates a possible embodiment wherein a gastric bandage 33 is applied.

The passage of the guide means through the walls of the tissues to be united can be obtained by perforating the wall (for example with radiofrequency needles) at the zone intended to form the anastomosis, so that after the formation of the anastomosis the continuity of the intestinal duct is restored.

In accordance with a possible embodiment, the guide means and the anastomotic device as previously described may be used in any technique, for example hybrid intraluminal and laparoscopic or other type.

In accordance with a possible embodiment, the instrumentation according to the present invention comprises means for closely approximating the tissue portions comprising an anastomotic device adapted to closely approximate and/or join the tissues intraluminally.

An anastomotic device adapted for such purpose can be a device adapted to form an anastomotic ring create the anastomosis, or a circular stapler sliding on the guide means and cooperating with an anvil, lockable on the guide means.

The instrumentation according to the present invention may also comprise means for partially creating a gastric pouch 23 before inserting the guide means, wherein the second tissue portion 25 of the gastric sleeve 21, 22 of stomach is arranged at the gastric pouch 23 thereof, or means for creating the complete gastric pouch 23 at the end of the procedure. In the first case, means are advantageously foreseen for completing the gastric pouch after the formation of the gastroenteroanastomosis and enteroenteroanastomosis.

The multistage method and the instrumentation described above with reference to a procedure with guide means, preferably intraluminally, may be applied both to the step of carrying out the gastroenteroanastomosis and the step of carrying out the enteroenteroanastomosis, or to only one of these steps of the gastrointestinal bypass phase of the procedure. As in the previously described embodiment, the method and instrumentation allow severely obese patients and obese patients with life threatening co-morbidity to become eligible for gastric bypass surgery and reduces the risks of mortality in gastrointestinal bypasses and considerably limits the operation times of the bypass phase. The maintenance of the continuity in the intestine until the completion of the two anastomoses permits the simultaneous verification of both. Moreover, due to the close arrangement of the two anastomoses, the operation area is limited to the upper zone of the abdomen.

While the present invention has been illustrated by description of several embodiments and while the illustrative embodiments have been described in considerable detail, it is not the intention to restrict or in any way limit the scope of the appended claims to such detail. Additional advantages and modifications may readily appear to those skilled in the art.

1. Instrumentation for carrying out a gastrointestinal bypass comprising:
means (4, 6, 12) for creating a gastric sleeve (21, 22) from the lesser curvature (13) of the stomach (1),
means for creating a gastrointestinal bypass involving said gastric sleeve (21, 22), in which said means for creating a gastrointestinal bypass include:
means for closely approximating a first tissue portion (24) of the intestine to a second tissue portion (25) of the gastric sleeve (21, 22) of stomach (1), creating a first loop (A) of the intestine between the gastric sleeve 21, 22 of stomach and the first tissue portion,
means for creating a gastroenteroanastomosis (26) between the two close tissue portions, maintaining the continuity of the intestinal duct after the creation of the gastroenteroanastomosis,
means for closely approximating an additional first tissue portion (27) of the intestine arranged distally or downstream with respect to the gastroenteroanastomosis (26) with reference to the natural flow along the intestinal duct, and an additional second tissue portion (28) of the intestine arranged proximally, or upstream, with respect to the gastroenteroanastomosis (26) with reference to the natural flow along the intestinal duct, creating a second loop (B) of the intestine which is distal with respect to the gastroenteroanastomosis (26), wherein said additional second tissue portion (28) of the intestine, proximal with respect to the gastroenteroanastomosis (26), is brought close to the gastroenteroanastomosis (26),
means for creating an enteroenteroanastomosis (29) between the two close tissue portions (27, 28) of the intestine, maintaining the continuity of the intestinal duct after the creation of the enteroenteroanastomosis (29),
means for sectioning the intestine (20) between the gastroenteroanastomosis (26) and the enteroenteroanastomosis (29).

2. Instrumentation according to claim 1, wherein the means for creating the gastroenteroanastomosis (26) comprise a linear stapler (6) adapted to partially join the flaps of a gastrotomy and enterotomy respectively made at the first portion (24) of the intestine and the second portion (25) of the gastric sleeve (21, 22) of stomach (1) and wherein the means for creating the enteroenteroanastomosis comprise a linear stapler adapted to partially join the flaps of enterotomies respectively made at the additional first and second portions of the intestine.

3. Instrumentation according to claim 1 or 2, comprising laparoscopic means adapted to perform the gastrointestinal bypass laparoscopically.

4. Instrumentation according to claim 1, comprising intraluminal means adapted to perform the gastrointestinal bypass intraluminally.

5. Method for carrying out a gastrointestinal bypass in a multistage procedure comprising the steps of:
creating a gastric sleeve (21, 22) from the lesser curvature (13) of the stomach (1),
creating a gastrointestinal bypass by means of the substeps:
closely approximating a first tissue portion (24) of the intestine to a second tissue portion (25) of the gastric sleeve (21, 22) of stomach (1), thereby creating a first loop (A) of the intestine between the gastric sleeve (21, 22) of stomach (1) and the first tissue portion (24),
creating a gastroenteroanastomosis (26) between the two close tissue portions (24,25), maintaining the continuity of the intestinal duct after the creation of the gastroenteroanastomosis (26),
closely approximating an additional first tissue portion (27) of the intestine arranged distally or downstream with respect to the gastroenteroanastomosis (26) with reference to the natural flow along the intestinal duct, and an additional second tissue portion (28) of the intestine arranged proximally, or upstream, with respect to the gastroenteroanastomosis (26) with reference to the natural flow along the intestinal duct, thereby creating a second loop (B) of the intestine (20) which is distal with respect to the gastroenteroanastomosis (26), wherein said additional second tissue portion (28) of the intestine (20), proximal with respect to the gastroenteroanastomosis (26), is brought close to the gastroenteroanastomosis (26),
creating an enteroenteroanastomosis (29) between the two close tissue portions (27, 28) of the intestine (20), maintaining the continuity of the intestinal duct after the creation of the enteroenteroanastomosis (29),
transecting the intestine (20) between the gastroenteroanastomosis (26) and the enteroenteroanastomosis (29).

6. Method according to claim 5, comprising a waiting phase between the creation of the gastric sleeve (21, 22) and the creation of the gastro-intestinal bypass, said waiting phase lasting at least 2 months, preferably 2 months to 3 years, yet more preferably 1 year to 2 years.

7. Method according to claim 5, wherein the intestine is sectioned between the gastroenteroanastomosis (26) and the enteroenteroanastomosis (29) after a step of simultaneously seal testing both the gastroenteroanastomosis (26) and the enteroenteroanastomosis (29).

8. Method according to one of the claims from 5 to 7, wherein the step of creating the gastroenteroanastomosis (26) is at least partially performed using a linear stapler, wherein one jaw of the linear stapler is inserted in a gastrotomy made at the second tissue portion of the gastric sleeve (21, 22) of stomach (1) and the other jaw of the linear stapler is inserted in an enterotomy made at the first tissue portion (24) of the intestine (20), to create at least one sequence of points which partially defines the gastroenteroanastomosis,
and wherein said linear stapler is used for bringing close together the two tissue portions to be joined, inserting one jaw of the linear stapler in an enterotomy made at the first tissue portion (24) of the intestine and transporting, by means of said linear stapler, the first intestine portion (24) in correspondence with a gastrotomy made in the second portion (25) of the gastric sleeve (21, 22) of stomach, before joining the two tissue portions.

9. Method according to any one of claims 5 to 8, wherein the step of creating the enteroenteroanastomosis (29) is at least partially performed by using a linear stapler, wherein one jaw of the linear stapler is inserted in an enterotomy made at the additional first tissue portion of the intestine and the other jaw of the linear stapler is inserted in an enterotomy made at the additional second tissue portion of the intestine, to realise at least one sequence of points which partially defines the enteroenteroanastomosis,
and wherein said linear stapler is used for bringing close together the two tissue portions to be joined, inserting one jaw of the linear stapler in an enterotomy made at the additional first tissue portion (27) of the intestine and transporting, by means of said linear stapler, the additional first intestine portion (27) in correspondence with an enterotomy made in the additional second portion (28) of the intestine before joining the two tissue portions.

10. Method according to one of the claims from 5 to 9, wherein a preliminary step is foreseen for making a gastric pouch (23) and wherein said second tissue portion (25) of the gastric sleeve (21, 22) of stomach (1) is arranged at the gastric pouch.

11. Method according to anyone of claims from 5 to 10, performed laparoscopically.

12. Method according to claim 11, performed with natural orifice assistance.

13. Method according to claim 5, wherein the gastrointestinal bypass phase is performed intraluminally.
